(19) United States
(12) Patent Application Publication
(10) Pub. No.: US 2025/0205184 A1
(43) Pub. Date: Jun. 26, 2025

(54) COLD COMPRESS SPRAY FOR RELIEVING GOUT AND PREPARATION METHOD THEREOF

(71) Applicant: Changde Jizhi Biological Technology Co., Ltd, Changde (CN)

(72) Inventor: Xinqiao HE, Changde (CN)

(21) Appl. No.: 18/391,872

(22) Filed: Dec. 21, 2023

(51) Int. Cl. A61K 31/205; A61K 9/00; A61K 9/12; A61K 36/068; A61K 36/28; A61K 36/52; A61K 36/63; A61K 36/886; A61P 19/06; A61P 29/00

(52) U.S. Cl. CPC A61K 31/205; A61K 9/0014; A61K 9/12; A61K 36/068; A61K 36/28; A61K 36/52; A61K 36/63; A61K 36/886; A61P 19/06; A61P 29/00

(57) ABSTRACT

A cold compress spray for relieving gout and preparation method thereof are provided. The cold compress spray for relieving gout is made from alkaline ionized water, baking soda, L-carnitine, Eucommia ulmoides bark water extract, olive juice, an aloe vera leaf, walnut shell, and Cordyceps sinensis. The cold compress spray of is a medicine for external use. Adverse reactions and side effects of the cold compress spray are obviously reduced. The cold compress spray has good penetrability, and can immediately decompose tophus and promote blood circulation after using hands to apply the cold compress spray to a gout area, thereby relieving pains. The disclosure has obvious treatment effects, obviously improve the medication compliance of gout patients, and improves life qualities of the gout patients.

### TECHNICAL FIELD

The disclosure relates to the technical field of medicine, and specifically to a cold compress spray for relieving gout and preparation method thereof.

### BACKGROUND

Gout is a characteristic arthritis caused by deposition of uric acid crystals in joints and tissues around joints, while the deposition of uric acid crystals in joints and tissues around joints is caused by purine metabolism disorder and the increase of blood uric acid. Clinically, gout is characterized by hyperuricemia, recurrent acute arthritis and gouty stone formation. Severe gout can lead to joint deformity and dysfunction, acute obstructive nephropathy or gouty nephropathy. At present, an incidence rate of gout in general population of China is 0.84%, with about 12 million people, of which 95% are male patients, and a ratio of male to female is about 20:1. There are about 120 million cases of hyperuricemia in China (accounting for about 10% of the total population), with a high incidence age of 50-59 years in males and an incidence rate in menopausal females (estrogen helps to excrete uric acid). In recent years, the incidence rate of gout has increased year by year due to the improvement of the living standard of people and changes of diets. With continuous research on the pathogenesis of gout, many gout treatment drugs have also been discovered in the research.

Traditional Chinese medicine in China has performed long-term observation and research on gout, and has rich clinical treatment experience. Through literature review, some scholars and medical scientists believe that gout has symptoms of “Lijie disease” described in Synopsis of the Golden Chamber, which are characterized by “shar pain, swollen feet, and inability to bend and stretch”. Gout has similar symptoms with acute arthritis. Through long-term clinical verification, aconite decoction and cassia twig-common peony-anemarrhena asphodeloides decoction have been proven to be effective in treating gout. But the vast majority of modern scholars believe that gout belongs to the category of arthralgia syndrome in traditional Chinese medicine, with a syndrome type being heat arthralgia or wetness-heat syndrome. Guozhang Hong, a modern renowned physician, proposed that there are two main causes of gout: the first cause is that external cold moisture invades the body; and the second cause is that gout patients have a dietary preference for alcohol and meat.

At present, traditional treatment methods for gout in China are divided into two types: traditional Chinese medicine and Western medicine. Western medicine mainly uses analgesics, anti-inflammatory drugs, and injections to temporarily relieve pain, only relieving symptoms rather than curing the root cause, and its side effects are significant. The treatment of traditional Chinese medicine is diversified, including syndrome differentiation, dietotherapy, traditional Chinese patent medicines and simple preparations, external application and washing, acupuncture and moxibustion, massage, acupoint injection, ear acupuncture and other treatments, with little side effects. However, traditional Chinese medicine generally suffers from shortcomings such as low concentration of active ingredients in medicinal materials, slow onset of action, and difficulty in achieving quick curative effect. The existing traditional Chinese medicine purification technology often leads to the loss or reduction of active ingredients after concentration and the decline of action effect after purification and concentration of such traditional Chinese medicines. Therefore, the development of a Chinese medicine extract with high concentration of active ingredients and rapid action has become a major problem and hot spot in current scientific research.

### SUMMARY

A purpose of the disclosure is to provide a cold compress spray for relieving gout and preparation method thereof. The cold compress spray of the disclosure is a medicine for external use. Adverse reactions and side effects of the cold compress spray are obviously reduced. The cold compress spray has good penetrability, and can immediately decompose tophus and promote blood circulation after using hands to apply the cold compress spray to a gout area, thereby relieving pains. The disclosure has obvious treatment effects, obviously improves the medication compliance of gout patients, and improves life qualities of the gout patients.

Compared with related art, the disclosure provides the following technical solutions:
a cold compress spray for relieving gout is provided, and raw materials of the cold compress spray include: alkaline ionized water, baking soda, L-carnitine, Eucommia ulmoides bark water extract, olive juice, an aloe vera leaf, walnut shell, and Cordyceps sinensis.

In an embodiment, the alkaline ionized water has 97-99 parts by weight; the baking soda has 0.2-1.8 parts by weight; the L-carnitine has 0.1-0.5 parts by weight; the Eucommia ulmoides bark water extract has 0.1-0.5 parts by weight; the olive juice has 0.01-0.1 parts by weight; the aloe vera leaf has 0.1-0.5 parts by weight; the walnut shell has 0.1-1 parts by weight; and the Cordyceps sinensis has 0.2-1 parts by weight.

In an embodiment, the alkaline ionized water has 97 parts by weight; the baking soda has 0.2 parts by weight; the L-carnitine has 0.1 parts by weight; the Eucommia ulmoides bark water extract has 0.1 parts by weight; the olive juice has 0.01 parts by weight; the aloe vera leaf has 0.1 parts by weight; the walnut shell has 0.1 parts by weight; and the Cordyceps sinensis has 0.2 parts by weight.

In an embodiment, the alkaline ionized water has 98 parts by weight; the baking soda has 1.0 parts by weight; the L-carnitine has 0.3 parts by weight; the Eucommia ulmoides bark water extract has 0.3 parts by weight; the olive juice has 0.05 parts by weight; the aloe vera leaf has 0.3 parts by weight; the walnut shell has 0.5 parts by weight; and the Cordyceps sinensis has 0.6 parts by weight.

In an embodiment, the alkaline ionized water has 99 parts by weight; the baking soda has 1.8 parts by weight; the L-carnitine has 0.5 parts by weight; the Eucommia ulmoides bark water extract has 0.5 parts by weight; the olive juice has 0.1 parts by weight; the aloe vera leaf has 0.5 parts by weight; the walnut shell has 1 part by weight; and the Cordyceps sinensis has 1 part by weight.

A preparation method for the cold compress spray for relieving gout is provided, and the preparation method includes:
step 1, adding the baking soda into the alkaline ionized water, and stirring evenly to obtain a soda alkaline solution with a potential of hydrogen (pH) value of 12-13;
step 2, adding the L-carnitine into the soda alkaline solution, and stirring evenly and standing to obtain a first solution;
step 3, adding the Eucommia ulmoides bark water extract and the olive juice into the first solution obtained in the step 2 to obtain a second solution, filtering the second solution to obtain a third solution;
step 4, crushing the Cordyceps sinensis into Cordyceps sinensis particles with a particle size corresponding to more than 100 meshes; and using the Cordyceps sinensis particles to prepare a Cordyceps sinensis solution;
step 5, weighting the walnut shell, soaking the walnut shell in purified water for 30-60 hours, placing the walnut shell into an extraction tank after soaking, using water with a weight 10-20 times a weight of the walnut shell as a solvent to extract the walnut shell to obtain an initial walnut shell solution, and filtering the initial walnut shell solution to obtain a walnut shell solution;
step 6, cleaning the aloe vera leaf, performing high-temperature instant sterilization on the aloe vera leaf, removing a base and a tip of the aloe vera leaf, cutting the aloe vera leaf with the base and tip removed into pieces to obtain slices, placing the slices in a container and pressing the slices while placing the slices in the container; repeating a process for 2-3 times, the process including freezing the slices for 6-8 hours outdoors and thawing the slices after the slices are frozen totally, to separate aloe vera pulp from aloe vera shells of the slices naturally, and filtering the aloe vera pulp to obtain aloe vera juice; and
step 7, concentrating the third solution, the Cordyceps sinensis solution, the aloe vera juice, and the walnut shell solution under a temperature of 25 Celsius degrees (° C.) to obtain a concentrated solution, and placing the concentrated solution into a spray filling device to obtain the cold compress spray

In an embodiment, a temperature of the freezing in the step 6 is −10° C. to −30° C.

In an embodiment, a relative density of the concentrated solution in the step 7 is 1.10-1.15.

The disclosure has the following beneficial effects.
(1) The cold compress spray of the disclosure is a medicine for external use. Adverse reactions and side effects of the cold compress spray are obviously reduced. The cold compress spray has good penetrability, and can immediately decompose tophus and promote blood circulation after using hands to apply the cold compress spray to a gout area, thereby relieving pains. The disclosure has obvious treatment effects, obviously, improves the medication compliance of gout patients, and improves life qualities of the gout patients.
(2) By adding aloe vera in the cold compress spray for relieving gout, since the aloe vera has effects of sterilization, inflammation diminishing, heat absorption and detumescence, the disclosure can effectively relieve swelling and pains caused by gout inflammation, thereby improving the curative effect of the cold compress spray for relieving gout.

### DETAILED DESCRIPTION OF EMBODIMENTS

In order to enable those skilled in the art to better understand technical solutions of the disclosure, the following is a clear and complete description of the technical solutions in embodiments of the disclosure. Obviously, the described embodiments are only a part of embodiments of the disclosure, not all of them. Based on the embodiments of the disclosure, all other embodiments obtained by those skilled in the art without creative work shall fall within the scope of protection of the disclosure.

### Embodiment 1

A cold compress spray for relieving gout and preparation method thereof are provided, and the preparation method includes:
step 1, adding 0.2 kilograms (kg) of baking soda into alkaline ionized water, and stirring evenly to obtain a soda alkaline solution with a pH value of 12;
step 2, adding 0.1 kg of L-carnitine into 99 liters (L) of the soda alkaline solution obtained in the step 1, and stirring evenly and standing to obtain a first solution;
step 3, adding 0.1 kg of Eucommia ulmoides bark water extract and 0.01 kg of olive juice into the first solution obtained in the step 2 to obtain a second solution, filtering the second solution to obtain a third solution;
step 4, crushing 0.2 kg of Cordyceps sinensis into Cordyceps sinensis particles with a particle size corresponding to more than 100 meshes; and using the Cordyceps sinensis particles to prepare a Cordyceps sinensis solution;
step 5, weighting 0.1 kg of walnut shell, soaking the walnut shell in purified water for 30 hours, placing the walnut shell into an extraction tank after soaking, using water with a weight 10 times a weight of the walnut shell as a solvent to extract the walnut shell to obtain an initial walnut shell solution, and filtering the initial walnut shell solution to obtain a walnut shell solution;
step 6, cleaning 0.1 kg of an aloe vera leaf, performing high-temperature instant sterilization on the aloe vera leaf, removing a base and a tip of the aloe vera leaf, cutting the aloe vera leaf with the base and tip removed into pieces to obtain slices, placing the slices in a container and pressing the slices while placing the slices in the container; repeating a process for 2 times, the process including freezing the slices for 8 hours outdoors under a temperature of −10° C. and thawing the slices after the slices are frozen totally, to separate aloe vera pulp from aloe vera shells of the slices naturally, and filtering the aloe vera pulp to obtain aloe vera juice; and
step 7, concentrating the third solution, the Cordyceps sinensis solution, the aloe vera juice, and the walnut shell solution under a temperature of 25° C. to obtain a concentrated solution with a relative density of 1.10, and placing the concentrated solution into a spray filling device to obtain the cold compress spray.

### Embodiment 2

A cold compress spray for relieving gout and preparation method thereof are provided, and the preparation method includes:
step 1, adding 1.8 kg of baking soda into 97 kg of alkaline ionized water, and stirring evenly to obtain a soda alkaline solution with a pH value of 12.5;
step 2, adding 0.5 kg of L-carnitine into the soda alkaline solution obtained in the step 1, and stirring evenly and standing to obtain a first solution;
step 3, adding 0.5 kg of Eucommia ulmoides bark water extract and 0.1 kg of olive juice into the first solution obtained in the step 2 to obtain a second solution, filtering the second solution to obtain a third solution;
step 4, crushing 0.2 kg of Cordyceps sinensis into Cordyceps sinensis particles with a particle size corresponding to more than 100 meshes; and using the Cordyceps sinensis particles to prepare a Cordyceps sinensis solution;
step 5, weighting 0.1 kg of walnut shell, soaking the walnut shell in purified water for 30-60 hours, placing the walnut shell into an extraction tank after soaking, using water with a weight 15 times a weight of the walnut shell as a solvent to extract the walnut shell to obtain an initial walnut shell solution, and filtering the initial walnut shell solution to obtain a walnut shell solution;
step 6, cleaning 0.1 kg of an aloe vera leaf, performing high-temperature instant sterilization on the aloe vera leaf, removing a base and a tip of the aloe vera leaf, cutting the aloe vera leaf with the base and tip removed into pieces to obtain slices, placing the slices in a container and pressing the slices while placing the slices in the container; repeating a process for 2 times, the process including freezing the slices for 7 hours outdoors under a temperature of −20° C. and thawing the slices after the slices are frozen totally, to separate aloe vera pulp from aloe vera shells of the slices naturally, and filtering the aloe vera pulp to obtain aloe vera juice; and
step 7, concentrating the third solution, the Cordyceps sinensis solution, the aloe vera juice, and the walnut shell solution under a temperature of 25° C. to obtain a concentrated solution with a relative density of 1.15, and placing the concentrated solution into a spray filling device to obtain the cold compress spray.

### Embodiment 3

A cold compress spray for relieving gout and preparation method thereof are provided, and the preparation method includes:
step 1, adding 1.0 kg of baking soda into 98 kg of alkaline ionized water, and stirring evenly to obtain a soda alkaline solution with a pH value of 13;
step 2, adding 0.2 kg of L-carnitine into the soda alkaline solution obtained in the step 1, and stirring evenly and standing to obtain a first solution;
step 3, adding 0.4 kg of Eucommia ulmoides bark water extract and 0.04 kg of olive juice into the first solution obtained in the step 2 to obtain a second solution, filtering the second solution to obtain a third solution;
step 4, crushing 0.8 kg of Cordyceps sinensis into Cordyceps sinensis particles with a particle size corresponding to more than 100 meshes; and using the Cordyceps sinensis particles to prepare a Cordyceps sinensis solution;
step 5, weighting 0.4 kg of walnut shell, soaking the walnut shell in purified water for 30-60 hours, placing the walnut shell into an extraction tank after soaking, using water with a weight 20 times a weight of the walnut shell as a solvent to extract the walnut shell to obtain an initial walnut shell solution, and filtering the initial walnut shell solution to obtain a walnut shell solution;
step 6, cleaning 0.4 kg of an aloe vera leaf, performing high-temperature instant sterilization on the aloe vera leaf, removing a base and a tip of the aloe vera leaf, cutting the aloe vera leaf with the base and tip removed into pieces to obtain slices, placing the slices in a container and pressing the slices while placing the slices in the container; repeating a process for 3 times, the process including freezing the slices for 8 hours outdoors under a temperature of −30° C. and thawing the slices after the slices are frozen totally, to separate aloe vera pulp from aloe vera shells of the slices naturally, and filtering the aloe vera pulp to obtain aloe vera juice; and
step 7, concentrating the third solution, the Cordyceps sinensis solution, the aloe vera juice, and the walnut shell solution under a temperature of 25° C. to obtain a concentrated solution with a relative density of 1.15, and placing the concentrated solution into a spray filling device to obtain the cold compress spray.

### Comparative Embodiment 1

A cold compress spray for relieving gout and preparation method thereof are provided, and the preparation method includes:
step 1, adding 1.0 kg of baking soda into 98 kg of alkaline ionized water, and stirring evenly to obtain a soda alkaline solution with a pH value of 13;
step 2, adding 0.2 kg of L-carnitine into the soda alkaline solution obtained in the step 1, and stirring evenly and standing to obtain a first solution;
step 3, crushing 0.8 kg of Cordyceps sinensis particles with a particle size corresponding to more than 100 meshes; and using the Cordyceps sinensis particles to prepare a Cordyceps sinensis solution;
step 4, weighting 0.4 kg of walnut shell, soaking the walnut shell in purified water for 30-60 hours, placing the walnut shell into an extraction tank after soaking, using water with a weight 20 times a weight of the walnut shell as a solvent to extract the walnut shell to obtain an initial walnut shell solution, and filtering the initial walnut shell solution to obtain a walnut shell solution;
step 5, cleaning 0.4 kg of an aloe vera leaf, performing high-temperature instant sterilization on the aloe vera leaf, removing a base and a tip of the aloe vera leaf, cutting the aloe vera leaf with the base and tip removed into pieces to obtain slices, placing the slices in a container and pressing the slices while placing the slices in the container; repeating a process for 3 times, the process including freezing the slices for 8 hours outdoors under a temperature of −30° C. and thawing the slices after the slices are frozen totally, to separate aloe vera pulp from aloe vera shells of the slices naturally, and filtering the aloe vera pulp to obtain aloe vera juice; and
step 6, concentrating the first solution, the Cordyceps sinensis solution, the aloe vera juice, and the walnut shell solution under a temperature of 25° C. to obtain a concentrated solution with a relative density of 1.15, and placing the concentrated solution into a spray filling device to obtain the cold compress spray.

### Comparative Embodiment 2

A cold compress spray for relieving gout and preparation method thereof are provided, and the preparation method includes:
step 1, adding 1.0 kg of baking soda into 98 kg of alkaline ionized water, and stirring evenly to obtain a soda alkaline solution with a pH value of 13;
step 2, adding 0.4 kg of Eucommia ulmoides bark water extract and 0.04 kg of olive juice into the soda alkaline solution obtained in the step 2 to obtain a first solution, filtering the first solution to obtain a second solution;
step 3, crushing 0.8 kg of Cordyceps sinensis into Cordyceps sinensis particles with a particle size corresponding to more than 100 meshes; and using the Cordyceps sinensis particles to prepare a Cordyceps sinensis solution;
step 4, weighting 0.4 kg of walnut shell, soaking the walnut shell in purified water for 30-60 hours, placing the walnut shell into an extraction tank after soaking, using water with a weight 20 times a weight of the walnut shell as a solvent to extract the walnut shell to obtain an initial walnut shell solution, and filtering the initial walnut shell solution to obtain a walnut shell solution;
step 5, cleaning 0.4 kg of an aloe vera leaf, performing high-temperature instant sterilization on the aloe vera leaf, removing a base and a tip of the aloe vera leaf, cutting the aloe vera leaf with the base and tip removed into pieces to obtain slices, placing the slices in a container and pressing the slices while placing the slices in the container; repeating a process for 3 times, the process including freezing the slices for 8 hours outdoors under a temperature of −30° C. and thawing the slices after the slices are frozen totally, to separate aloe vera pulp from aloe vera shells of the slices naturally, and filtering the aloe vera pulp to obtain aloe vera juice; and
step 6, concentrating the second solution, the Cordyceps sinensis solution, the aloe vera juice, and the walnut shell solution under a temperature of 25° C. to obtain a concentrated solution with a relative density of 1.15, and placing the concentrated solution into a spray filling device to obtain the cold compress spray.

### Test 1: The Cold Compress Spray of the Disclosure is Applied to Acute Plantar Swelling of Rat Caused by Sodium Urate Crystals

A process of preparing sodium urate crystallization and a sodium urate solution is as follows: 2 g of uric acid are added into 400 mL of boiling water to obtain an uric acid solution; a pH value of the uric acid solution is adjusted to 7.4 by using NaOH (1N) to obtain an adjusted solution; the adjusted solution is heated to a temperature of 95° C. to obtain a heated solution; then the heated solution is cooled and stirred gently at a room temperature to obtain a cooled solution; the cooled solution is allowed to stand for 4-6 hours at 4° C. to precipitate flocculent precipitates; microcrystalline sodium urate is obtained by filtering, and the microcrystalline sodium urate is sterilized at a high temperature of 80° C. 1000 milligrams (mg) of the sodium urate are ground fully to obtain sodium urate powder, and the sodium urate powder is added into 20 mL of sterile physiological saline to prepare a suspension with a concentration of 50 g/L (5%).

A process of grouping and administration is as follows: rats are adaptively fed for 3 days and randomly divided into 7 groups based on body weights. The 7 groups include a model control group, a positive drug indomethacin group, a first group, a second group, and a third group, a first comparison group, and a second comparison group. One hour before inflammation, the positive drug indomethacin group is administered with indomethacin through gavage (10 mL/kg). The first group is immediately administered with the cold compress spray (0.1 mL for each rat) obtained in the embodiment 1 after modeling; the second group is immediately administered with the cold compress spray (0.1 mL for each rat) obtained in the embodiment 2 after modeling; the third group is immediately administered with the cold compress spray (0.1 mL for each rat) obtained in the embodiment 3 after modeling; the first comparison group is immediately administered with the cold compress spray (0.1 mL for each rat) obtained in the comparative embodiment 1 after modeling; the second comparison group is immediately administered with the cold compress spray (0.1 mL for each rat) obtained in the comparative embodiment 2 after modeling; and the model control group is immediately administered with an excipient (0.1 mL for each rat) after modeling. Furthermore, after inflammation, the first group, the second group, the third group, the first comparison group, and the second comparison group are administered with corresponding cold compress sprays again after measurements of joint swelling at 6, 12, and 24 hours.

A process of modeling is as follows: an area around an ankle joint of a right hind limb of each rat is marked with a marker pen, and a joint circumference is measured as a normal value using a plantar volume meter. 0.2 mL of 5% sodium urate suspension are injected (the joint of each rat is extended and a needle is inserted at 45 degrees) into a joint cavity on a dorsal side of the right ankle joint of each rat of each group. Here, the modeling of each group is completed.

Results of joint swelling are as follows: a volume of the right ankle joint of each rat at 6, 12, 24, and 48 hours after inflammation is measured. And degrees of joint swelling of each group are as shown in TABLE 1.

$$Degree of joint swelling = volume of joint before modeling - volume of joint after modeling .$$

TABLE 1
effects of sodium urate on ankle joint swelling of rats (X ± SD, n = 9)

| | | Volume | | | | |
|---|---|---|---|---|---|---|
| | | before | | | | |
| | | modeling | Volume of after modeling (mL) | | | |
| Group | Dosage | (mL) | 6 h | 12 h | 24 h | 48 h |
| Model control | 0.1 mL | 2.09 ± 0.14 | 2.84 ± 0.29 | 2.97 ± 0.31 | 2.72 ± 0.34 | 2.33 ± 0.30 |
| group | for each | | | | | |
| | rat | | | | | |
| Indomethacin | 0.0045 | 2.12 ± 0.21 | 2.42 ± 0.15 | 2.29 ± 0.30 | 2.18 ± 0.09 | 2.16 ± 0.14 |
| | g/kg | | | | | |
| First group | 0.1 mL | 2.0 ± 0.15 | 2.51 ± 0.23 | 2.43 ± 0.24 | 2.22 ± 0.31 | 2.13 ± 0.15 |
| | for each | | | | | |
| | rat | | | | | |
| Second group | 0.1 mL | 2.04 ± 0.13 | 2.62 ± 0.17 | 2.47 ± 0.18 | 2.29 ± 0.19 | 2.15 ± 0.12 |
| | for each | | | | | |
| | rat | | | | | |
| Third group | 0.1 mL | 2.02 ± 0.20 | 2.49 ± 0.43 | 2.49 ± 0.42 | 2.39 ± 0.24 | 2.14 ± 0.19 |
| | for each | | | | | |
| | rat | | | | | |
| First | 0.1 mL | 2.04 ± 0.09 | 2.72 ± 0.30 | 2.87 ± 0.20 | 2.60 ± 0.20 | 2.26 ± 0.22 |
| comparison | for each | | | | | |
| group | rat | | | | | |
| Second | 0.1 mL | 1.98 ± 0.14 | 2.74 ± 0.28 | 2.85 ± 0.20 | 2.64 ± 0.20 | 2.32 ± 0.25 |
| comparison | for each | | | | | |
| group | rat | | | | | |

Conclusion: For the model control group, after 6 hours, the ankle joint volume of rats significantly increased, and the joint swelling gradually decreased with time; however, it still did not return to normal after 48 hours, and there is a significant difference between the joint swelling before inflammation and the joint swelling after inflammation (P<0.01). Compared with the model control group, the first group, the second group, and the third group significantly reduce ankle joint volumes of rats (P<0.05, P<0.01). For the first comparison group and the second comparison group, there is no improvement of joint swelling in the first comparison group and the second comparison group, and therefore there is no statistical significance compared to the model control group.

### Test 2: Analgesic Test of the Cold Compress Spray of the Disclosure

A hot plate method is used in the test. Female mice weighing 20-24 g are selected for the test, and a multi-purpose instrument for pharmacological and physiological experiments is used to adjust a temperature to 55° C.±0.5. A pain index is determined according to the mice licking hind feet. A pain threshold value is measured every 5 minutes (min) before administration, two pain threshold values are measured in total, and an average value of the two pain threshold values is taken as a normal pain threshold value. Mice having the normal pain threshold value between 10-30 seconds, stable response, and no jumping are selected for the test. 10 mice are set in each group, and groups for the test include:
a basic group, administered with 70% ethanol solution;
a first group, administered with the cold compress spray obtained in the embodiment 1;
a second group, administered with the cold compress spray obtained in the embodiment 2;
a third group, administered with the cold compress spray obtained in the embodiment 3;
a first comparison group, administered with the cold compress spray obtained in the comparison embodiment 1; and
a second comparison group, administered with the cold compress spray obtained in the comparison embodiment 2.

Both sides of hind feet of each mouse in each group are applied with 15 μL of corresponding medicine every 15 minutes, three times in total. After the last application of medicine, a first pain threshold value of each mouse is measured at 15 minutes, 30 minutes, and 60 minutes. After 5 minutes, a second pain threshold value of each mouse is also measured under the same condition as that of the first pain threshold value. Then, an average value of the first pain threshold value and the second pain threshold value is taken as a resultant pain threshold value. The results are shown in TABLE 2.

TABLE 2
Effects of medicines on pain threshold values of mice

| | | Pain threshold value (S, X ± SD) | | | |
|---|---|---|---|---|---|
| | | Before | | | |
| | | adminis- | After administration | | |
| Group | Amount | tration | 15 min | 30 min | 60 min |
| Basic | 10 | 9.4 ± 1.8 | 9.4 ± 2.6 | 9.0 ± 1.9 | 9.0 ± 1.8 |
| group | | | | | |
| First | 10 | 10.6 ± 2.3 | 21.8 ± 2.3 | 19.8 ± 2.5 | 19.5 ± 2.0 |
| group | | | | | |
| Second | 10 | 10.5 ± 2.9 | 22.3 ± 1.6 | 19.4 ± 2.3 | 19.5 ± 1.0 |
| group | | | | | |
| Third | 10 | 10.7 ± 1.3 | 22.4 ± 0.9 | 21.3 ± 1.6 | 21.5 ± 1.9 |
| group | | | | | |
| First | 10 | 9.9 ± 2.3 | 10.8 ± 2.8 | 12.5 ± 2.6 | 12.3 ± 2.2 |
| comparison | | | | | |
| group | | | | | |
| Second | 10 | 10.6 ± 2.6 | 11.6 ± 1.8 | 12.6 ± 2.3 | 12.4 ± 2.0 |
| comparison | | | | | |
| group | | | | | |

Conclusion: the local application of the cold compress spray of the disclosure can improve the pain threshold value caused by the hot plate method in mice. The cold compress spray of the comparison embodiments cannot improve the pain threshold value of mice, which fully shows that the composition of the disclosure is not a simple combination, but an overall organic combination.

What is claimed is:

1. A cold compress spray for relieving gout, wherein the cold compress spray is made from: alkaline ionized water, baking soda, L-carnitine, Eucommia ulmoides bark water extract, olive juice, an aloe vera leaf, walnut shell, and Cordyceps sinensis.

2. The cold compress spray for relieving gout as claimed in claim 1, wherein the alkaline ionized water has 97-99 parts by weight; the baking soda has 0.2-1.8 parts by weight; the L-carnitine has 0.1-0.5 parts by weight; the Eucommia ulmoides bark water extract has 0.1-0.5 parts by weight; the olive juice has 0.01-0.1 parts by weight; the aloe vera leaf has 0.1-0.5 parts by weight; the walnut shell has 0.1-1 parts by weight; and the Cordyceps sinensis has 0.2-1 parts by weight.

3. The cold compress spray for relieving gout as claimed in claim 1, wherein the alkaline ionized water has 97 parts by weight; the baking soda has 0.2 parts by weight; the L-carnitine has 0.1 parts by weight; the Eucommia ulmoides bark water extract has 0.1 parts by weight; the olive juice has 0.01 parts by weight; the aloe vera leaf has 0.1 parts by weight; the walnut shell has 0.1 parts by weight; and the Cordyceps sinensis has 0.2 parts by weight.

4. The cold compress spray for relieving gout as claimed in claim 1, wherein the alkaline ionized water has 98 parts by weight; the baking soda has 1.0 parts by weight; the L-carnitine has 0.3 parts by weight; the Eucommia ulmoides bark water extract has 0.3 parts by weight; the olive juice has 0.05 parts by weight; the aloe vera leaf has 0.3 parts by weight; the walnut shell has 0.5 parts by weight; and the Cordyceps sinensis has 0.6 parts by weight.

5. The cold compress spray for relieving gout as claimed in claim 1, wherein the alkaline ionized water has 99 parts by weight; the baking soda has 1.8 parts by weight; the L-carnitine has 0.5 parts by weight; the Eucommia ulmoides bark water extract has 0.5 parts by weight; the olive juice has 0.1 parts by weight; the aloe vera leaf has 0.5 parts by weight; the walnut shell has 1 part by weight; and the Cordyceps sinensis has 1 part by weight.

6. A preparation method for the cold compress spray for relieving gout as claimed in claim 1, comprising:
step 1, adding the baking soda into the alkaline ionized water, and stirring evenly to obtain a soda alkaline solution with a potential of hydrogen (pH) value of 12-13;
step 2, adding the L-carnitine into the soda alkaline solution, and stirring evenly and standing to obtain a first solution;
step 3, adding the Eucommia ulmoides bark water extract and the olive juice into the first solution obtained in the step 2 to obtain a second solution, filtering the second solution to obtain a third solution;
step 4, crushing the Cordyceps sinensis into Cordyceps sinensis particles with a particle size corresponding to more than 100 meshes; and using the Cordyceps sinensis particles to prepare a Cordyceps sinensis solution;
step 5, weighting the walnut shell, soaking the walnut shell in purified water for 30-60 hours, placing the walnut shell into an extraction tank after soaking, using water with a weight 10-20 times a weight of the walnut shell as a solvent to extract the walnut shell to obtain an initial walnut shell solution, and filtering the initial walnut shell solution to obtain a walnut shell solution;
step 6, cleaning the aloe vera leaf, performing high-temperature instant sterilization on the aloe vera leaf, removing a base and a tip of the aloe vera leaf, cutting the aloe vera leaf with the base and tip removed into pieces to obtain slices, placing the slices in a container and pressing the slices while placing the slices in the container; repeating a process for 2-3 times, the process comprising freezing the slices for 6-8 hours outdoors and thawing the slices after the slices are frozen totally, to separate aloe vera pulp from aloe vera shells of the slices naturally, and filtering the aloe vera pulp to obtain aloe vera juice; and
step 7, concentrating the third solution, the Cordyceps sinensis solution, the aloe vera juice, and the walnut shell solution under a temperature of 25 Celsius degrees (° C.) to obtain a concentrated solution, and placing the concentrated solution into a spray filling device to obtain the cold compress spray.

7. The preparation method as claimed in claim 6, wherein a temperature of the freezing in the step 6 is −10° C. to −30° C.

8. The preparation method as claimed in claim 6, wherein a relative density of the concentrated solution in the step 7 is 1.10-1.15.

9. The preparation method as claimed in claim 6, wherein in the step 6, time for freezing the slices outdoors is 8 hours.

10. The preparation method as claimed in claim 6, wherein in the step 1, the soda alkaline solution has the pH value of 12.

11. The preparation method as claimed in claim 6, wherein in the step 1, the soda alkaline solution has the pH value of 12.5.

12. The preparation method as claimed in claim 6, wherein in the step 5, the water with the weight 10 times the weight of the walnut shell is used as the solvent to extract the walnut shell.

13. The preparation method as claimed in claim 6, wherein in the step 5, the water with the weight 20 times the weight of the walnut shell is used as the solvent to extract the walnut shell.

14. The preparation method as claimed in claim 6, wherein in the step 5, the water with the weight 15 times the weight of the walnut shell is used as the solvent to extract the walnut shell.

15. The preparation method as claimed in claim 8, wherein the relative density of the concentrated solution 1.15.

16. The preparation method as claimed in claim 8, wherein the relative density of the concentrated solution 1.10.

17. The preparation method as claimed in claim 7, wherein a temperature of the freezing in the step 6 is −30° C.

18. The preparation method as claimed in claim 7, wherein a temperature of the freezing in the step 6 is −20° C.

19. A preparation method for the cold compress spray for relieving gout as claimed in claim 1, comprising:
step 1, adding the baking soda into the alkaline ionized water to obtain a soda alkaline solution;
step 2, adding the L-carnitine into the soda alkaline solution to obtain a first solution;
step 3, adding the Eucommia ulmoides bark water extract and the olive juice into the first solution obtained in the step 2 to obtain a second solution, filtering the second solution to obtain a third solution;
step 4, crushing the Cordyceps sinensis into Cordyceps sinensis particles; and using the Cordyceps sinensis particles to prepare a Cordyceps sinensis solution;
step 5, weighting the walnut shell, soaking the walnut shell in purified water, placing the walnut shell into an extraction tank after soaking, using water as a solvent to extract the walnut shell to obtain an initial walnut shell solution, and filtering the initial walnut shell solution to obtain a walnut shell solution;
step 6, processing the aloe vera leaf to obtain aloe vera juice; and
step 7, concentrating the third solution, the Cordyceps sinensis solution, the aloe vera juice, and the walnut shell solution to obtain a concentrated solution, and placing the concentrated solution into a spray filling device to obtain the cold compress spray.

20. The preparation method as claimed in claim 19, wherein in the step 7, a relative density of the concentrated solution is 1.10-1.15.
